(19) United States
(12) Patent Application Publication
(10) Pub. No.: US 2026/0215833 A1
(43) Pub. Date: Jul. 30, 2026

(54) ABLATION ZONE VISUALIZATION FOR MULTIPLE PROBES

(71) Applicant: Neuwave Medical, Inc., Madison, WI (US)

(72) Inventor: Matthew SCHANING, Cambridge, WI (US)

(73) Assignee: Neuwave Medical, Inc., Madison, WI (US)

(21) Appl. No.: 19/038,225

(22) Filed: Jan. 27, 2025

(51) Int. Cl. A61B 18/00; A61B 34/00; A61B 34/10

(52) U.S. Cl. CPC A61B 18/00; A61B 34/10; A61B 34/25; A61B 2018/00577; A61B 2018/00702; A61B 2018/00761; A61B 2034/104; A61B 2034/107

(57) ABSTRACT

A system is disclosed that includes a first ablation probe including a first antenna, a second ablation probe including a second antenna, and a controller in operable communication with a display. The controller is operable to receive operating parameters associated with the first and second ablation probes, display, on the display, an image of the first and second antennas in a patient, determine a distance between the first and second antennas, compare the distance to a distance threshold, determine an angle between the first and second antennas, compare the angle to an angle threshold, and select a number of expected ablation zones to overlay on the displayed image based on the operating parameters and the comparisons.

### BACKGROUND

The present disclosure is related to systems and methods for delivering energy to tissue for an ablation operation and, more particularly, to systems and methods for visualizing expected ablation treatments using one or more ablation probes.

During a surgical procedure, a clinician may identify a target tissue (e.g. a lesion or tumor) that may be too large to feasibly ablate with one ablation probe. Accordingly, the clinician may position more than one ablation probe (e.g. two ablation probes) at or near the target tissue to co-operatively ablate the same. However, the clinician may not know if the co-operative efforts of the multiple ablation probes are sufficient to ablate the target tissue.

Accordingly, systems and methods for displaying expected, or predicted, ablation zones that are expected to be generated by ablation probes are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system including an energy delivery device and a display, in accordance with at least one aspect of the present disclosure.

FIG. 2 is a schematic diagram of the energy delivery device of FIG. 1, in accordance with at least one aspect of the present disclosure.

FIG. 3 is an enlarged, cross-sectional view of a portion of the energy delivery device of FIG. 2, in accordance with at least one aspect of the present disclosure.

FIGS. 4A and 4B are a flow diagram for determining a number of expected, or predicted, ablation zones to overlay on a display, according to at least one aspect of the present disclosure.

FIG. 5 is the display of FIG. 1 displaying images according to a first example implementation of the flow diagram of FIGS. 4A and 4B, according to at least one aspect of the present disclosure.

FIG. 6 is the display of FIG. 1 displaying images according to a second example implementation of the flow diagram of FIGS. 4A and 4B, according to at least one aspect of the present disclosure.

### DETAILED DESCRIPTION

The present disclosure is related to systems and methods for delivering energy to tissue for an ablation operation and, more particularly, to systems and methods for visualizing expected ablation treatments using multiple ablation probes.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

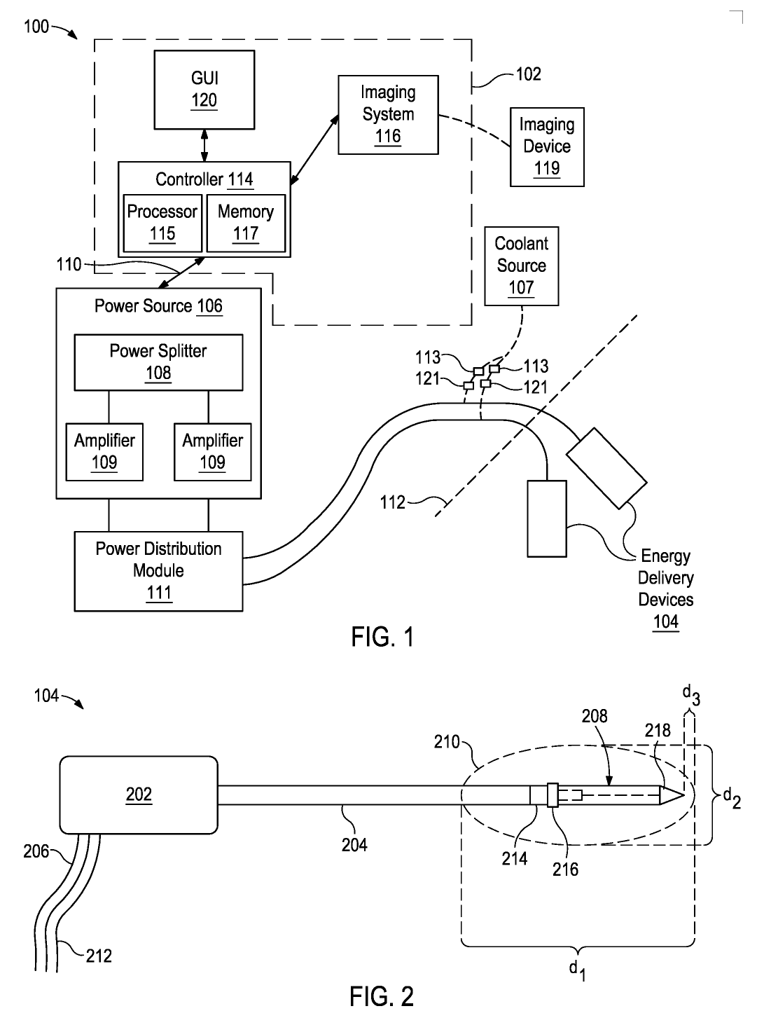

FIG. 1 is a block diagram of an energy delivery system 100, in accordance with at least one aspect of the present disclosure. As illustrated, the energy delivery system 100 (hereafter “the system 100”) may include a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes).

The system 100 may further include a power source or generator 106 communicably coupled to the control system 102 and the ablation probes 104 to direct, control, and deliver (provide) electrical power thereto. The power source 106 may include a power splitter 108 that receives power from an external power source (e.g. a wall outlet) and directs power to one or more amplifiers 109 (two shown), which may amplify the voltage, current, or power from the power splitter 108 to an associated ablation probe 104. While two amplifiers 109 are shown, each associated with a corresponding ablation probe 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which may provide strain relief to cabling extending from the amplifiers 109 to the ablation probes 104. The power distribution module 111 may be coupled to a structure in the operating room, such as a surgical bed, and may house connection hardware of the probes 104.

The power source 106 may supply energy required to operate various components of the system 100. The power source 106 may also supply energy to the ablation probes 104, such as microwave energy, radiofrequency energy, radiation, cryo energy, electroporation, high intensity focused ultrasound, or any combination thereof. In accordance with principles of the present disclosure, the power source 106 may supply microwave energy to the ablation probes 104 for purposes of tissue ablation. More specifically, power may be supplied to the ablation probes 104, but the microwave energy may be generated in a microwave generator and sent to the ablation probe 104. The power source 106 may include one or more energy generators configured to provide as much as 140-150 watts of microwave power at a frequency from 915 MHz to 5.8 GHz, although the present disclosure is not so limited. The power splitter 108 may comprise a power distribution system operable to distribute the energy from the power source 106 to the ablation probes 104. The power splitter 108 may be configured to provide varying energy levels to different regions of the ablation probes 104.

The control system 102 may monitor, control, and provide feedback concerning operation of the system 100. As illustrated, the control system 102 may include a controller 114, an imaging system 116, and a graphical user interface (GUI) or display 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature, as discussed in more detail below.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected (expected) ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

The imaging system 116 may be in communication with the controller 114 and comprise one or more imaging devices 119. Example imaging devices include, but are not limited to, ultrasound transducers, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

The imaging system 116 may be configured to monitor ablation procedures, such as a position of the ablation probes 104 within a patient, as described in more detail below, and/or the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The components of the system 100 may be connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The system 100 may further include a coolant source 107, which stores therein a cooling fluid or “coolant”. Example coolants include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

The system 100 may further include one or more valves 113 fluidically coupled to the coolant source 107 and a respective ablation probe 104. The valves 113 may control the flow rate and/or pressure of coolant from the coolant source 107 to the respective ablation devices 104. The valves 113 may be any suitable valve (e.g., gate, globe, ball, etc.) that is transitionable (actuatable) between an open state (e.g., 100% open), a closed state (e.g.,0% open), and a plurality of partially open states between the open and closed states (e.g.,10%, 25%, 50%, 75%, or 90%). In applications where the valves 113 are electromechanically actuatable, the valves 113 may each include a motor in operable communication with the controller 114, via a wired or wireless connection, and which function to transition the valves 113 between their respective open, closed, and partially open states. Alternatively, the valves 113 may be solenoid valves that are transitionable between their respective open, closed, and partially opened states by the controller 114. The valves 113 may be transitionable between their respective open, closed, and partially open states based on a user input provided to the GUI 120 or automatically, such as based on inputs provided to the controller 114 from various sensors of the system 100.

The system 100 may further include one or more sensors 121 for sensing one or more parameters associated with the coolant provided from the coolant source 107. The sensors 121 may include pressure sensors operable to sense a pressure of the coolant provided from the coolant source 107, flow sensors operable to sense a flow rate of the coolant provided from the coolant source 107, or temperature sensors for sensing a temperature of the coolant provided from the coolant source 107, or any combination thereof. Each valve 113 may have associated therewith one or more of the sensors 121 to sense one or more parameters of the coolant provided to the respective ablation probe 104. The controller 114 may be in operable communication with the one or more sensors 121, via a wired or wireless connection, and may receive the sensed parameters therefrom. The controller 114 may control one or more aspects of the system 100 based on the sensed parameters, such as the state of the valves 113.

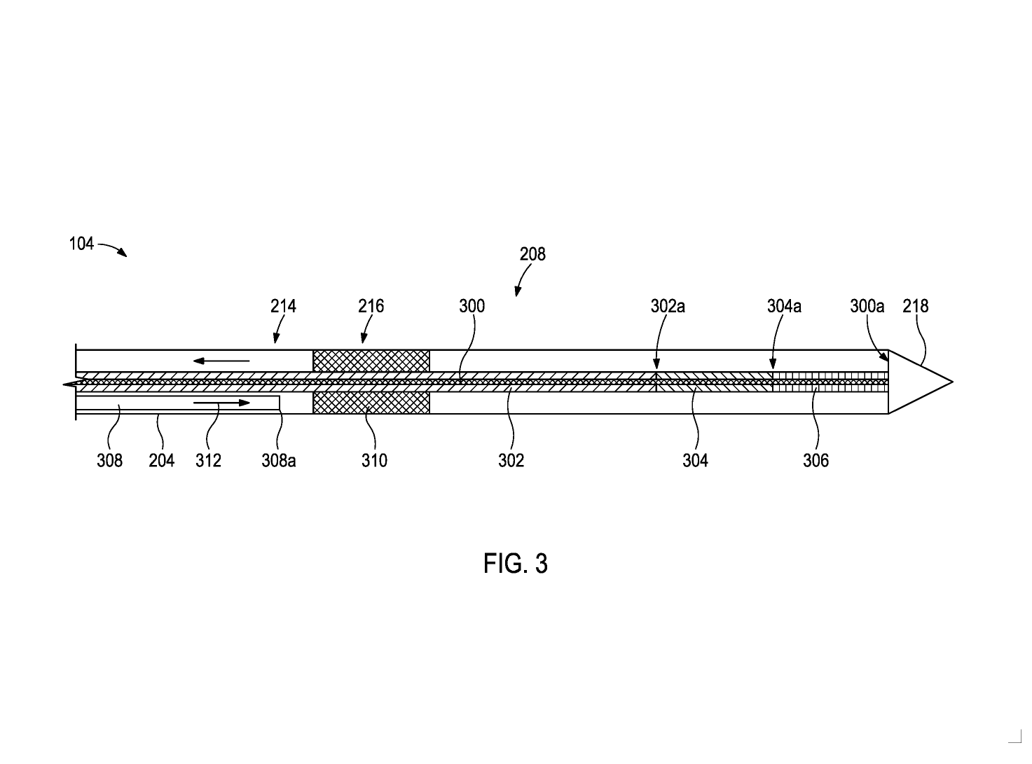

FIG. 2 is a schematic diagram of an example ablation probe 104, in accordance with at least one aspect of the present disclosure. As indicated above, the ablation probe 104 is configured to deliver (emit) energy (e.g., microwave energy, radiofrequency energy, radiation energy) to a target tissue region. As illustrated, the ablation probe 104 includes a handle or housing 202 and an elongate shaft or probe cannula 204 extending distally from the handle 202.

A cable or cable assembly 206 is operatively coupled to the handle 202 and configured to convey electrical power thereto. The cable assembly 206 may extend from the power distribution module 111 (FIG. 1), for example, and provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210 (shown in dashed lines).

The ablation zone 210 includes a longitudinal length d₁, a lateral width d₂(e.g. a diameter of the ablation zone 210), and a distance d₃ between a distal end of the stylet 218 and a distal-most end of the ablation zone 210. The size of the dimensions d₁, d₂, d₃ of the ablation zone 210 depend on one or more operating parameters, such as the type of ablation probe, the power level P of the power source 106, and the amount of time T the ablation probe 104 is energized. For instance, a first type of ablation probe 104 that is energized at a first power level P₁ for a first amount of time T₁ may be expected to generate an ablation zone 210 that includes a first longitudinal length (d₁), a first lateral width (d₂) and a first distance (d₃) between the distal end of the stylet 218 and the distal-most end of the ablation zone, while a second ablation probe 104 different than the first ablation probe that is energized at a second power level P₂ different than the first power level P₁ for a second amount of time T₂ different than the first amount of time T₁ may be expected to generate an ablation zone that includes a second longitudinal length (d₁’) different from the first longitudinal length (d₁), a second lateral width (d₂’) different from the first lateral width (d₂), and a second distance (d₃’) between the distal end of the stylet 218 and the distal-most end of the ablation zone different from the first distance (d₃). It should be noted that only one of the parameters described above (e.g. type of instrument, power level, and time) may need to be adjusted to change the resulting dimensions d₁, d₂, d₃.

The memory 117 may store therein a look-up table that includes various combinations of types of ablation probes, activation times T, and power levels P that are expected to yield various sized ablation zones 210 with varying dimensions (e.g. d₁, d₂, d₃). The expected, or predicted, dimensions of the ablation zone may be based on ex-vivo data, in-vivo data, or clinical data, or combinations thereof. Accordingly, should a user desire to generate an ablation zone 210 with particular dimensions, the controller 114 retrieves, from the memory 117, a particular combination of type of ablation probe, activation time, and power level that is expected to yield an ablation zone that meets, or at least substantially comes close to, the dimensions desired of the user. Additional information regarding predicted ablation zones is discussed in U.S. Patent Application No. 18/811,266, entitled “DYNAMIC VISUALIZATION OF EXPECTED ABLATION ZONE”, filed August 21, 2024, which is hereby incorporated by reference in its entirety herein.

A cooling tube 212 is operatively coupled to the coolant source 107 (FIG. 1), and the handle 202 and is configured to convey the coolant from the coolant source 107 to the ablation probe 104.

The ablation probe 104 includes a sharp stylet tip or “stylet” 218 located at the distal end of the antenna 208 and otherwise forming the distal end of the ablation probe 104. The stylet 218 facilitates percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be brazed to zirconia or an equivalent of zirconia. In such applications, the stylet 218 may comprise an extension of a metal portion of the antenna 208 and may be electrically active.

The ablation probe 104 may further include a stick region 214, alternately referred to as a “tissue-loc” region, provided on the probe cannula 204 and a plug region 216 provided on the probe cannula 204 distal to the stick region 214 at or near the antenna 208. The stick and plug regions 214, 216 may be defined as portions of the probe cannula 204, and will be discussed in more detail below.

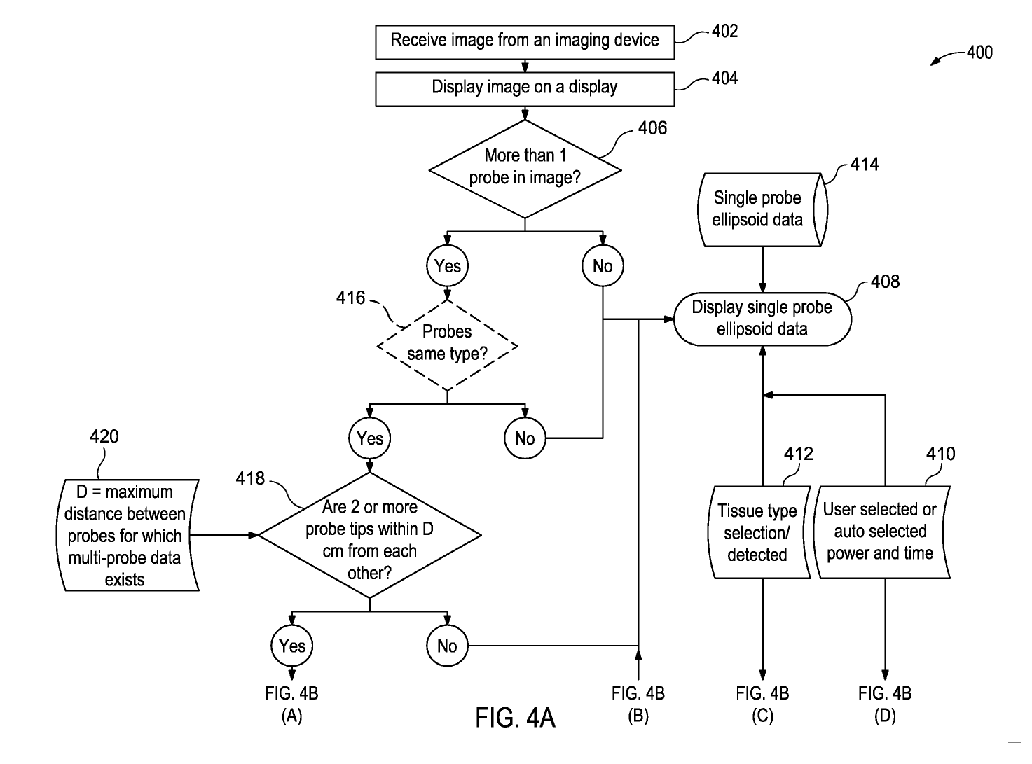

FIG. 3 is an enlarged, cross-sectional view of the probe cannula 204 and antenna 208 of the ablation probe 104, in accordance with at least one aspect of the present disclosure. The ablation probe 104 includes an inner conductor 300 and an outer conductor 302 extending through the probe cannula 204 and the antenna 208, with the outer conductor 302 being positioned about (around) the inner conductor 300 (i.e., the inner conductor 300 extends within the outer conductor 302). As illustrated, a distal end 300a of the inner conductor 300 extends distal to (beyond) a distal end 302a of the outer conductor 302. The inner and outer conductors 300, 302 are made of a conductive material that allows current to be transmitted along their lengths thereof. The inner and outer conductors 300, 302 may be made of a metal, for example, such as stainless steel, silver, copper, brass or aluminum, or alloys thereof.

The ablation probe 104 further includes an insulator 304 extending from the distal end 302a of the outer conductor 302 and positioned about (around) the inner conductor 300. The insulator 304 may be made of a variety of non-conductive materials including, but not limited to, a ceramic or a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The ablation probe 104 further includes a conductor load 306 extending from a distal end 304a of the insulator 304 and positioned about (around) the inner conductor 300. Accordingly, the insulator 304 axially interposes the outer conductor 302 and the conductor load 306. The conductor load 306 may be made of a metal, such as stainless steel, silver, copper, brass or aluminum, alloys thereof, or any combination thereof. The conductor load 306 may serve as a load point and may include a longitudinal length that is tuned to match the dielectric properties of the surrounding tissue.

The ablation probe 104 further includes a cooling tube 308 extending through the probe cannula 204 and terminating at the stick region 214. The cooling tube 308 fluidly communicates with the cooling tube 212 (FIG. 2) to convey coolant 312 from the coolant source 107 (FIG. 1) and the cooling tube 212 to the stick region 214. A user may provide an input to the handle 202 (FIG. 2) and/or the GUI 120 (FIG. 1) to control flow of the coolant 312 through the cooling tube 308 to regulate a temperature of the antenna 208, the ablation zone 210, and/or the stick region 214, as discussed in more detail below. The cooling tube 308 may be made of a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The stick region 214 is designed to attain and maintain a temperature that promotes adherence of a tissue region onto the surface of the stick region 214. More specifically, the stick region 214 operates as an anchoring element that freezes the interface between the stick region 214 and the adjacent tissue, thereby sticking (maintaining, locking, etc.) the antenna 208 in place. In operation, coolant 312 from the coolant source 107 (FIG. 1) is conveyed to the stick region 214 by way of the cooling tube 212 (FIG. 2) and expelled from a distal end 308a of the cooling tube 308. Upon expulsion from the distal end 308a of the cooling tube 308, the pressure of the coolant 312 quickly decreases, thereby causing the coolant 312 to correspondingly decrease in temperature (the Joule-Thompson effect). The decreased temperature of the coolant 312 causes the temperature of the stick region 214 to decrease, and the coolant 312 is recirculated back along the probe cannula 204, such as to a coolant sink, for example.

Once a threshold “low” temperature is reached at the stick region 214, contact with adjacent tissue causes the tissue to adhere (stick or couple) to the stick region 214, thereby resulting in attachment of the energy delivery device 104 to the tissue. During ablation, as the tissue warms, the antenna 208 remains secured to the tissue region due to tissue desiccation and charring. The stick region 214 may be made of any material able to attain and maintain a temperature such that contact with tissue results in adherence of the tissue onto the stick region 214. Example materials for the stick region 214 include, but are not limited to, a metal.

With reference now to FIGS. 1 and 3, the controller 114 may control a state of one or more of the valves 113, thereby controlling an amount of coolant 312 provided to the stick region 214 of the ablation probe 104. Tissue may not adhere to the stick region 214 unless a threshold flow rate and/or pressure of coolant 312 is conveyed through the cooling tube 308 to the stick region 214. For instance, the valves 113 are transitionable between a first state and a second state. The first state may be a first, partially opened state of the valve 113, where the valve 113 is opened a first amount, and the second state may be a second, partially opened state or the open state of the valve 113, where the valve 113 is opened a second amount greater than the first amount. The first and second states of the valves 113 may be stored in the memory 117.

In the first state, a first flow rate and/or pressure of coolant 312 is conveyed through the cooling tube 308 to the stick region 214. The first flow rate and/or pressure of coolant 312 may be insufficient to cause tissue to adhere to the stick region 214, thereby allowing the stick region 214 to move relative to the tissue positioned thereagainst. In the second state, a second flow rate and/or pressure of coolant 312 greater than the first flow rate and/or pressure of coolant 312 is conveyed through the cooling tube 308 to the stick region 214. The second flow rate and/or pressure of coolant 312 may be sufficient to cause tissue to adhere to the stick region 214, thereby preventing the stick region 214 from moving relative to the tissue positioned thereagainst.

The ablation probe 104 may further include a seal 310, which may define the plug region 216 on the probe cannula 204. As illustrated, the seal 310 is provided distal to the distal end 308a of the cooling tube 308 and the stick region 214 and otherwise interposing the stick region 214 and the antenna 208. The seal 310 is configured to prevent a reduction in temperature resulting from the cooled probe cannula 204 and the stick region 214 from affecting (e.g., reducing) the temperature within the antenna 208. Accordingly, the seal 310 separates interior portions of the ablation probe 104 to prevent cooling or heating of a portion or portions of the probe 104 while permitting cooling or heating of other portions. The seal 310 may be made of an insulative material capable of being in contact with a material or region having a low temperature without having its temperature significantly reduced. Example insulative materials for the seal 310 include, but are not limited to, a synthetic polymer (e.g., polystyrene, polyicynene, polyurethane, polyisocyanurate), aerogel, fiberglass, cork, or any combination thereof.

Additional information regarding the ablation probe 104, such as the construction and function thereof, is described in U.S. Patent No. 11,638,607, entitled “ENERGY DELIVERY SYSTEMS AND USES THEREOF”, which issued on May 2, 2023, the contents of which are hereby incorporated by reference in their entirety herein.

With continued reference to FIGS. 1 and 3, the controller 114 may be operable to use the coolant source 107 and the coolant stored therein to reduce undesired heating within and along the ablation probes 104. In particular, the controller 114 may control conveyance (flow) of the coolant into and out of the cooling tube 308 via the valves 113. The controller 114 may also be configured to control conveyance of coolant to the stick region 214, as described herein above to thereby attain and maintain a temperature that accommodates adherence of tissue onto the surface of the stick region 214. In some embodiments, a user may provide an input to the controller 114, such as via the GUI 120. Based on the input, the controller 114 may control a state of one or more of the valves 113, as discussed herein above, thereby allowing the coolant source 107 to provide coolant to the stick region 214 via the cooling tubes 212, 308.

The controller 114 may also be operable to continuously or intermittently monitor the real-time temperature of the ablation probes 104. In such embodiments, the controller 114 communicates with one or more temperature sensors (e.g., thermocouples) terminating at various points along the probe cannula 204 and/or the antenna 208 (FIG. 2) of the ablation probe 104. Consequently, localized temperature is monitored at several points along the antenna 208 to estimate ablation status, cooling status, or safety checks. In some applications, monitoring the temperature at several points along the antenna 208 helps determine the geographical characteristics of the ablation zone 210, such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

In some embodiments, the probe cannula 204 includes a plurality of temperature sensors. A first temperature sensor may be placed at, or slightly proximal to, the antenna 208 to provide real-temperature measurements of the tissue being heated by the antenna 208. A second temperature sensor may be placed at, or adjacent to, the stick region 214 to provide real-time temperature measurements of the tissue that is being cooled, and thus adhered to, the stick region 214. A third temperature sensor may be located proximal to the first and second temperature sensors along the cannula 204, such as at the point of entry into the skin, to provide real-time measurements of the patent’s skin. The control system 102 can receive the temperature measurements from the first, second, and third sensors to control the coolant systems and cooling fluids from the controller 114 to the stick region 214 and/or other cooling systems of the energy delivery device 104.

The controller 114 may also be operable to monitor the temperature of a tissue region (e.g., tissue being treated, surrounding tissue). This may prove advantageous in helping to determine the status of the procedure (e.g., the end of the procedure). The controller 114 may communicate with the plurality of temperature sensors to provide real-time temperature information to a user and display such measurements on the GUI 120. In at least one embodiment, based on the temperature data obtained by the controller 114, the controller 114 may be configured to autonomously adjust operation of the system 100 appropriately.

### Ablation Zone Visualization for Multiple Probes

During a surgical procedure, a clinician may identify a target tissue (e.g. a lesion or tumor) that may be too large to feasibly ablate with one ablation probe 104 without repositioning the ablation probe 104 multiple times. In such cases, the clinician may introduce and position two or more ablation probes 104 at or near the target tissue to co-operatively ablate the same. However, the clinician may not know if the co-operative efforts of the multiple ablation probes are sufficient to ablate the target tissue. Accordingly, systems and methods for displaying expected, or predicted, ablation zones that are expected to be generated by ablation probes are desired.

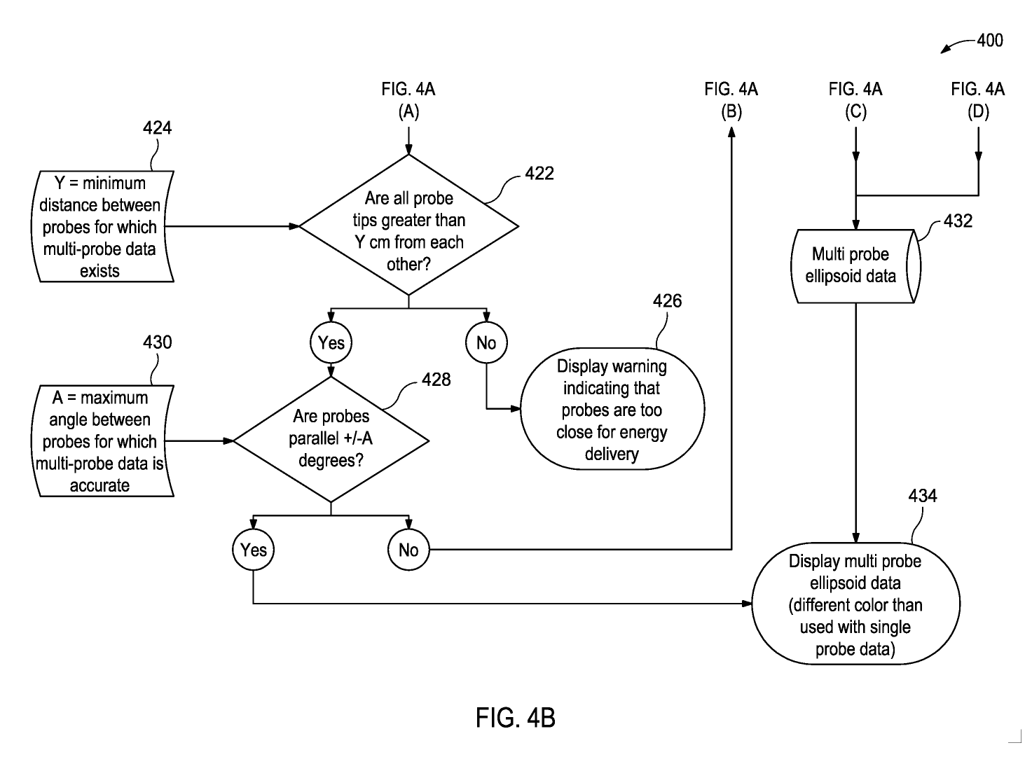

FIGS. 4A and 4B depict a schematic flowchart of an example method 400 for determining a number of expected, or predicted, ablation zones to overlay on a display, according to at least one aspect of the present disclosure. The method 400 may be embodied as an algorithm, stored in the memory 117 (FIG. 1) of the controller 114 (FIG. 1), and may be executed by the processor 115 (FIG. 1), such as based on an input provided to the controller 114 (FIG. 1) by a user.

With reference to FIGS. 1, 4A, and 4B, the method 400 may include receiving an image from an imaging device, as at step 402. For instance, the imaging device 119 may capture (obtain) an image of a patient, such as an MRI image, a CT image, an ultrasound image, a nuclear medicine image, or a fluoroscopy image. The imaging device 119 may capture the image based on a user providing an input to the controller 114, which then receives the image from the imaging device 119.

The method 400 may further include displaying the image on a display, as at step 404. For instance, based on receiving the image from the imaging device 119, the controller 114 may display, on the display 120, the image for a user to visualize.

The method 400 may further include determining a number of ablation probes present in the image, as at step 406. The controller 114 may determine the number of ablation probes 104 present within the image by using image recognition software, based on a user input provided to the controller 114, based on a number of ablation probes 104 coupled to the power source 106, based on a number of ablation probes 104 coupled to the coolant source 107, or any combination thereof. From the number of ablation probes 104 present in the image, the controller 114 may be configured to determine if the number of ablation probes 104 is less than, equal to or greater than one.

If it is determined that no ablation probes are in the image at step 406, the method 400 may include providing an alert. For instance, if the controller 114 determines that there are no ablation probes in the image, the controller 114 may provide an alert to the user, such as via the display 120, and thereby informing the user that a new image is needed. In such cases, the method 400 may then proceed back to step 402.

If it is determined that there is only one ablation probe in the image at step 406, the method 400 may proceed to step 408, at which a single expected ablation zone may be overlaid on the display. More specifically, if the controller 114 determines that a single ablation probe 104 is present in the image, the controller 114 may proceed with overlaying a single ablation zone (e.g., an ellipse), like ablation zone 210 (FIG. 2), over the ablation probe 104 on the display 120.

The size and/or shape of the overlaid ablation zone may be based on one or more operating parameters 410 of the viewed ablation probe or a type of tissue 412 to be ablated by the ablation probe, or a combination thereof. The operating parameters 410 may include a power level, a duration of treatment (amount of time or “activation time”) at which to energize the ablation probe 104, the type of ablation probe, or combinations thereof, which may be provided to the controller 114 via an input interface, such as a touchscreen of the display 120. The type of tissue to be ablated may be provided to the controller 114 via an input interface, such as a touchscreen of the display 120, or may be detected and determined by the controller 114 using the imaging device 119 and image recognition software stored in the memory 117.

Based on the operating parameters 410 and the type of tissue 412, the controller 114 may overlay, on the image, a single ablation zone expected to be produced by the ablation probe 104. The memory 117 may have stored thereon data 414 that correlates set times, power levels, types of ablation probes, and tissue types to expected (predicted) dimensions for the ablation zone. The data may include ex-vivo data, in-vivo data, or clinical data, or combinations thereof. Like ablation zone 208 (FIG. 2), the expected ablation zone may be displayed on the display 120 with an expected longitudinal length d₁ (FIG. 2), an expected lateral width d₂ (FIG. 2), or an expected distance d₃ (FIG. 2) between a distal end of the stylet 218 (FIG. 2) and a distal-most end of the predicted ablation zone, or combinations thereof. Additional information regarding overlaying expected ablation zones is discussed in U.S. Patent Application No. 18/811,266, entitled “DYNAMIC VISUALIZATION OF EXPECTED ABLATION ZONE”, filed August 21, 2024, which is hereby incorporated by reference in its entirety herein. Accordingly, step 408 may yield an image that includes a single ablation probe and a single expected ablation zone.

If it is determined that there is more than one ablation probe in the image at step 406, the method 400 may optionally proceed to step 416, at which the types of the ablation probes visible in the image are determined and compared. More specifically, if the controller 114 determines that more than one ablation probe is present in the image (e.g. a first ablation probe and a second ablation probe), the controller 114 may then proceed with determining whether the ablation probes are the same or different types. In some cases, for instance, the first ablation probe may be expected to generate an ellipsoid or “tear-drop” shaped ablation zone, while the second ablation probe may be expected to generate a circular ablation zone. The types of the multiple ablation probes may be provided to the controller 114 by the user, such as via an input interface (e.g. a touchscreen of the display 120), or may be automatically detected by the controller 114, such as based on the ablation probes 104 being coupled to the controller 114 via the power source 106 or with image recognition software, or a combination thereof.

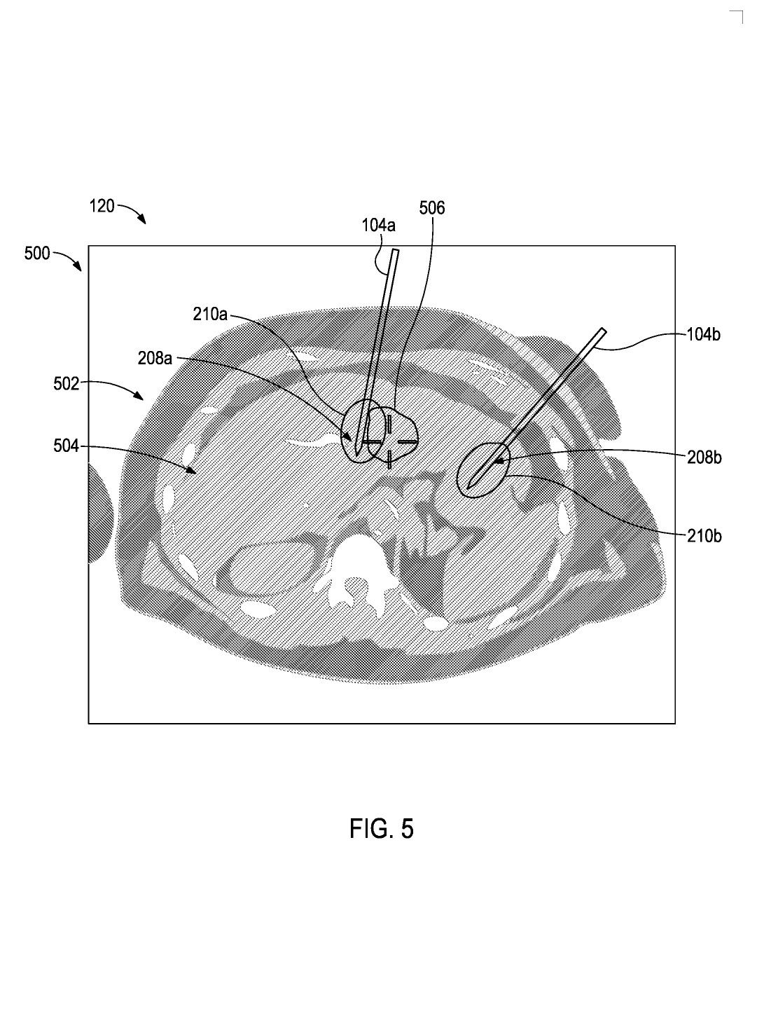

If it is determined that the ablation probes are different (e.g., the first ablation probe is a first type and the second ablation probe is a second type different than the first type), the method 400 may proceed to step 408, at which each of the visualized ablation probes in the image may be overlaid with its respective expected ablation zone, as described above with reference to step 408. Accordingly, the method 400 may yield an image that includes multiple ablation probes and multiple expected ablation zones; see, e.g., FIG. 5, which will be described in more detail below.

If it is determined that the ablation probes are the same type of ablation probe (e.g., the first and second ablation probes are the same type of ablation probe), the method 400 may proceed to step 418, at which a distance between the probes may be determined and compared to a first or “maximum” distance threshold 420. More particularly, if the controller 114 determines that there is more than one ablation probe in the image, the controller 114 may then be configured to determine a distance between the multiple ablation probes. The distance may be the distance between the antennas 208 (FIG. 2), the stylets 218 (FIG. 2), the seals 216 (FIG. 2), the stick regions 214 (FIG. 2), or the shafts 204 (FIG. 2), for example. The distance may be measured by the controller 114 using image recognition software or via sensors coupled to the ablation probes, such as Hall-Effect sensors, or a combination thereof. The first distance threshold may be stored in the memory 117 and retrievable by the processor 115, or may be provided to the controller 114 by a user, such as via a touchscreen of the display 120. In some embodiments, the first distance threshold may be about 5 centimeters, less than 5 centimeters (e.g., about1, 2, 3, or 4 centimeters), or greater than 5 centimeters (e.g., about 6, 7, 8, or 9 centimeters).

If it is determined that the distance between the ablation probes (e.g.,a distance between the first and second ablation probes) is at or greater than the first distance threshold, the method 400 may proceed to step 408, at which each of the visualized ablation probes in the image may be overlaid with its own respective, expected ablation zone, as described above. Accordingly, the method 400 may yield an image that includes multiple ablation probes and multiple expected ablation zones; see, e.g., FIG. 5.

If it is determined that the distance between the ablation probes (e.g.,a distance between the first and second ablation probes) is at or less than the first distance threshold, the method 400 may then proceed to step 422, at which the distance between the probes (e.g., distance determined at step 418) is compared to a second or “minimum” distance threshold 424. More specifically, if the controller 114 determines that the first and second ablation probes are positioned at or less than the first distance threshold, the controller 114 may compare the distance to the second distance threshold, which may be stored in the memory 117 and retrievable by the processor 115, or may be provided to the controller 114 by a user, such as via a touchscreen of the display 120. The second distance threshold may be about 0.1 centimeters, less than 0.1 centimeters (e.g.,about0.05, 0.075, or 0.09 centimeters), or greater than 0.1 centimeters (e.g., about 0.15, 0.25, or 0.5 centimeters).

If it is determined that the distance between the ablation probes (e.g.,the distance between the first and second ablation probes) is at or less than the second distance threshold, the method 400 may proceed to step 426, at which an alert or warning may be provided to a user. More specifically, if the measured distance is at or less that the second distance threshold, the controller 114 may generate an alert, such as an audible or visual alert via the display 120, or a tactile alert via the handle 202, thereby informing the user that the probes are too close together, which could result in arcing between the antennas. Based on the alert, the user may reposition one or more of the ablation probes and the method 400 may be restarted at step 402.

If it is determined that the distance between the ablation probes (e.g.,the distance between the first and second ablation probes) is at or greater than the second distance threshold, the method 400 may proceed to step 428, at which an angle between the probes may be determined and compared to a “maximum” angle threshold 430. More specifically, if the controller 114 determines that the ablation probes are at or greater than the second distance threshold, the controller 114 may then be configured to also determine an angle between the ablation probes. The angle may be measured relative to the antennas 208 (FIG. 2), the stylets 218 (FIG. 2), the seals 216 (FIG. 2), the stick regions 214 (FIG. 2), or the shafts 204 (FIG. 2), for example. The angle may be measured by the controller 114 using image recognition software that determines the probe positions in 3D space and determines the angle based on the determined positions or via sensors coupled to the ablation probes, such as Hall-Effect sensors, or a combination thereof. The angle threshold may be stored in the memory 117 and retrievable by the processor 115, or may be provided to the controller 114 by a user, such as via a touchscreen of the display 120. The maximum angle threshold may be about 45 degrees, less than 45 degrees (e.g.,about0, 10, 25, or 35 degrees), or greater than 45 degrees (e.g., about 50, 55, or 65 degrees).

If it is determined that the angle between the ablation probes (e.g.,an angle between the first and second ablation probes) is at or greater than the angle threshold, the method 400 may proceed to step 408, at which each of the visualized ablation probes in the image may be overlaid with its respective expected ablation zone. Accordingly, the method 400 may yield an image that includes multiple ablation probes and multiple expected ablation zones; see, e.g., FIG. 5.

If it is determined that the angle between the ablation probes (e.g.,an angle between the first and second ablation probes) is at or less than the angle threshold, the method 400 may proceed to step 432, at which a single “multi-probe” expected ablation zone may be overlaid on the display. More specifically, if the controller 114 determines that the angle between the first and second ablation probes is at or less than the angle threshold, the controller 114 may then be configured to generate and overlay a single multi-probe ablation zone over the first and second ablation probes 104 on the display 120.

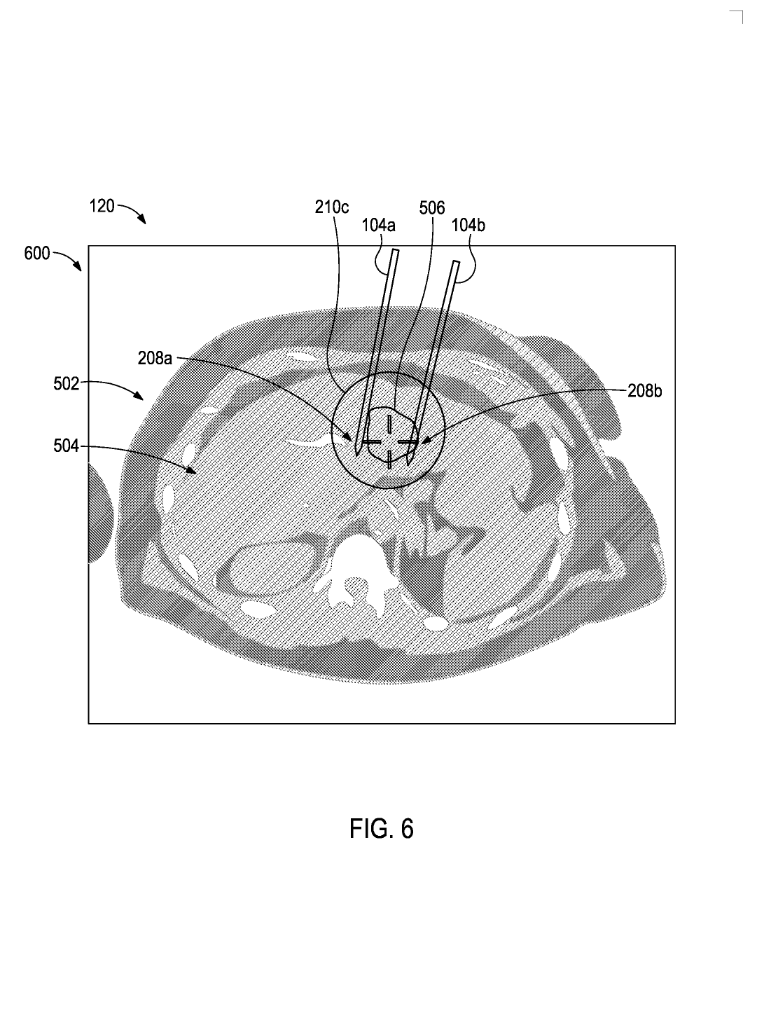

The method 400 may optionally include receiving a second image from the imaging device to determine if the probes are offset one another and/or to determine a second angle between the probes, such as after step 428 and prior to step 434. For instance, the image captured at step 402 may be a top-down view of the patient; see e.g., FIGS. 5 and 6. From this image, it may be difficult, or impossible, to determine if the probes are offset (e.g. vertically offset) relative to one another or if the probes are angled (e.g. “up” or “down”) relative to one another. Accordingly, the imaging device 119 may capture (obtain) an image of the patient, such as an MRI image, a CT image, an ultrasound image, a nuclear medicine image, or a fluoroscopy image, from an alternative point of view, such as a sideways point of view, to determine if the probes are offset (vertically) one another and/or to determine a second angle (e.g. “up” or “down” angle) between the probes. The offset between the probes and the second angle therebetween may be measured using image recognition software or sensors, similar to what was described elsewhere herein with respect to steps 418, 422, 428. An offset threshold and a second angle threshold may be stored in the memory 117 and may be retrievable by the processor 115, or may be provided to the controller 114 by a user, such as via a touchscreen of the display 120. The controller 114 may compare the determined offset and second angle to the respective offset threshold and second angle threshold.

If it is determined that the offset between the ablation probes (e.g.,the vertical offset between the first and second ablation probes) is at or greater than the offset threshold and/or that the second angle between the ablation probes (e.g.,the “up” and “down” angle between the first and second ablation probes) is at or greater than the second angle threshold, the method 400 may proceed to step 408, at which each of the visualized ablation probes in the image may be overlaid with its respective expected ablation zone, as described above with reference to step 408. If it is determined that the offset between the ablation probes (e.g.,the vertical offset between the first and second ablation probes) is at or less than the offset threshold and/or that the second angle between the ablation probes (e.g.,the “up” and “down” angle between the first and second ablation probes) is at or less than the second angle threshold, the method 400 may proceed to step 432, at which a single “multi-probe” expected ablation zone may be overlaid on the display, as described above.

The size and/or shape of the multi-probe expected ablation zone may be based on operating parameters 410 of the viewed ablation probes or a type of tissue 412 to be ablated by the ablation probes, or a combination thereof, as described herein above. Based on the operating parameters 410 and the type of tissue 412, the controller 114 may overlay on the image the multi-probe expected ablation zone that is expected to be generated (produced) by multiple ablation probes 104. The memory 117 may have stored thereon data that correlates set times, power levels, types of ablation probes, and tissue types to expected (predicted) dimensions for the multi-probe ablation zone. The data may include ex-vivo data, in-vivo data, or clinical data, or combinations thereof. Like ablation zone 208 (FIG. 2), the multi-probe expected ablation zone may be displayed with an expected longitudinal length d₁(FIG. 2), an expected lateral width d₂(FIG. 2), or an expected distance d₃ (FIG. 2) between a distal end of the stylet 218 (FIG. 2) and a distal-most end of the predicted ablation zone, or combinations thereof. Accordingly, the method 400 may yield an image that includes multiple ablation probes and a single expected ablation zone resulting from operation of the multiple ablation probes; see, e.g., FIG. 6, which is described in more detail below.

FIG. 5 depicts a first CT image 500 of a patient 502 presented on the display 120 of FIG. 1, and displaying images according to a first example implementation of the method 400 of FIGS. 4A-4B, according to at least one aspect of the present disclosure. The first CT image 500 of the patient 502 may be captured (obtained) by the imaging device 119 (FIG. 1) and displayed by the controller 114 (e.g.,during steps 402, 404 of the method 400). In particular, the CT image 500 shows a body cavity 504 of the patient 502 into which a first ablation probe 104a and a second ablation probe 104b have been inserted. The first ablation probe 104a is positioned adjacent an ablation target 506 (e.g., a target tissue to be ablated; a lesion or tumor), and the second ablation probe 104b is positioned in the body cavity 504 at a location spaced from the first ablation probe 104a and the ablation target 506, such as adjacent a separate (second) ablation target.

Implementing the method 400 described herein, the controller 114 determined that the first and second ablation probes 104a, 104b were spaced a distance apart that is greater than the first distance threshold (e.g.,step 418 of method 400), or that the first and second ablation probes 104a, 104b were angled relative to one another greater than the angle distance (e.g.,step 428 of method 400). As a result, as shown in FIG. 5, the controller 114 overlaid on the display 120 expected, or predicted, first and second ablation zones 210a and 210b that are expected to be generated by the first and second ablation probes 104a,b, respectively, and according to the operating parameters (step 410), the tissue type (step 412), and the single probe data (step 414).

FIG. 6 depicts a second CT image 600 of the patient presented on the display 120 of FIG. 1, and displaying images according to a second example implementation of the method 400 of FIGS. 4A and 4B, according to at least one aspect of the present disclosure. As with the first CT image 500 of FIG. 5, the second CT image 600 of the patient 502 may be captured (obtained) by the imaging device 119 (FIG. 1) and displayed by the controller 114 (e.g. during steps 402, 404 of the method 400). In particular, the CT image 600 shows the body cavity 504 of the patient 502 with the first ablation probe 104a having been maintained in the same position as in the first CT image 500 (FIG. 5) and the second ablation probe 104b repositioned adjacent the ablation target 506.

Implementing the method 400 described herein, the controller 114 determined that the first and second ablation probes 104a, 104b were spaced a distance apart that is less than the first distance threshold (e.g.,step 418 of method 400) and greater than the second distance threshold (e.g.,step 422 of method 400), as well as angled relative to one another less than the angle threshold (e.g.,step 428 of method 400). As a result, as shown in FIG. 6, the controller 114 overlaid on the display 120 a single “multi-probe” expected ablation zone 210c that is expected to be co-operatively generated by the first and second ablation probes 104a,b during operation, and according to the operating parameters (step 410), the tissue type (step 412), and the multi-probe data (step 432).

Accordingly, the foregoing systems and methods enable a user to visualize the size and shape of ablation zones that are expected (predicted) to be generated by two or more ablation probes.

### Embodiments disclosed herein include:

A. A system comprising a first ablation probe including a first antenna, a second ablation probe including a second antenna, and a controller in operable communication with a display and operable to receive operating parameters associated with the first and second ablation probes, display, on the display, images of the first and second antennas positioned within a patient, thereby resulting in a displayed image, determine a distance between the first and second antennas, compare the distance to a distance threshold, determine an angle between the first and second antennas, compare the angle to an angle threshold, and select a number of expected ablation zones to overlay on the displayed image based on the operating parameters and the comparisons.

B. A system comprising a first ablation probe including a first antenna, a second ablation probe including a second antenna, and a controller in operable communication with a display and operable to receive operating parameters associated with the first and second ablation probes, display, on the display, images of the first and second antennas in a patient, thereby resulting in a displayed image, compare a distance between the first and second antennas to a distance threshold, compare an angle between the first and second antennas to an angle threshold, and determine a number of predicted ablation zones to overlay on the displayed image based on the operating parameters and the comparisons.

C. A non-transitory computer readable medium storing instructions that, when executed by a processor, cause the processor to receive operating parameters associated with first and second ablation probes and second ablation probes, display, on the display, an image of the first and second antennas in a patient, determine a distance between the first and second antennas, compare the distance to a distance threshold, determine an angle between the first and second antennas, compare the angle to an angle threshold, and select a number of expected ablation zones to overlay on the displayed image based on the operating parameters and the comparisons.

Each of embodiments A, B, and C may have one or more of the following additional elements in any combination: Element 1: wherein the controller is further operable to overlay, on the displayed image, one expected ablation zone based on the distance being at or less than the distance threshold and the angle being at or less than the angle threshold. Element 2: wherein the controller is further operable to overlay, on the displayed image, the one expected ablation zone over the images of the first and second antennas. Element 3: wherein the controller is further operable to overlay, on the displayed image, two expected ablation zones based on the distance being at or greater than the distance threshold or the angle being at or greater than the angle threshold or a combination thereof. Element 4: wherein the controller is further operable to overlay, on the displayed image a first of the two expected ablation zones over the image of the first antenna and a second of the two expected ablation zones over the image of the second antenna. Element 5: wherein the controller is further operable to compare a type of the first ablation probe to a type of the second ablation probe and select the number of expected ablation zones to overlay on the displayed image based on the comparison of the types. Element 6: wherein the controller is further operable to overlay, on the displayed image, one expected ablation zone based on the distance being at or less than the distance threshold, the angle being at or less than the angle threshold, and the type of the first ablation probe being the same as the type of the second ablation probe. Element 7: wherein the controller is further operable to overlay, on the displayed image, two expected ablation zones based on the distance being at or greater than the distance threshold, the angle being at or greater than the angle threshold, or the type of the first ablation probe being different than the type of the second ablation probe, or combinations thereof. Element 8: wherein the distance threshold is a maximum distance threshold, and the controller is further operable to compare the distance to a minimum distance threshold less than the first distance threshold and provide an alert based on the distance being less than the minimum distance threshold. Element 9: wherein the operating parameters comprise an activation time and a power level of the first and second ablation probes. Element 10: wherein the controller is further operable to overlay, on the displayed image, one expected ablation zone based on the distance being at or less than the distance threshold and the angle being at or less than the angle threshold. Element 11: wherein the controller is further operable to overlay, on the displayed image, two expected ablation zones based on the distance being at or greater than the distance threshold or the angle being at or greater than the angle threshold or a combination thereof. Element 12: wherein the controller is further operable to compare a type of the first ablation probe to a type of the second ablation probe and determine the number of predicted ablation zones to overlay on the displayed image further based on the comparison of the types. Element 13: wherein the controller is further operable to overlay, on the displayed image, one expected ablation zone based on the distance being at or less than the distance threshold, the angle being at or less than the angle threshold, and the type of the first ablation probe being the same as the type of the second ablation probe. Element 14: wherein the controller is further operable to overlay, on the displayed image, two expected ablation zones based on the distance being at or greater than the distance threshold, the angle being at or greater than the angle threshold, or the type of the first ablation probe being different than the type of the second ablation probe, or combinations thereof. Element 15: wherein the operating parameters comprise an activation time and a power level of the first and second ablation probes. Element 16: further storing instructions that, when executed by the processor, cause the processor to overlay, on the displayed image, one expected ablation zone based on the distance being at or less than the distance threshold and the angle being at or less than the angle threshold. Element 17: further storing instructions that, when executed by the processor, cause the processor to overlay, on the displayed image, two expected ablation zones based on the distance being at or greater than the distance threshold or the angle being at or greater than the angle threshold or a combination thereof.

By way of non-limiting example, exemplary combinations applicable to A, B, and C include: Element 1 with Element 2; Element 3 with Element 4; Element 5 with Element 6; Element 5 with Element 7; Element 1 with one or more of Elements 2-9; Elements 1 and 2 with one or more of Elements 1 or 3-9; Element 3 with one or more of Elements 1, 2, or 4-9; Elements 3 and 4 with one or more of Elements 1, 2, or 5-9; Element 5 with one or more of Elements 1-4 or 6-9; Elements 5 and 6 with one or more of Elements 1-4 or 7-9; Elements 5 and 7 with one or more of Elements 1-4, 6, 8, or 9; Element 8 with one or more of Elements 1-7 or 9; Element 9 with one or more of Elements 1-8; Element 10 with one or more of Elements 11-15; Element 11 with one or more of Elements 10 or 12-15; Element 12 with one or more of Elements 10, 11, or 13-15; Element 12 with Element 13; Elements 12 and 13 with one or more of Elements 10, 11, 14, or 15; Element 12 with Element 14; Elements 12 and 14 with one or more of Elements 10, 11, 13, or 15; Element 15 with one or more of Elements 10-14; Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
a first ablation probe including a first antenna;
a second ablation probe including a second antenna; and
a controller in operable communication with a display and operable to:
receive operating parameters associated with the first and second ablation probes;
display, on the display, images of the first and second antennas positioned within a patient, thereby resulting in a displayed image;
determine a distance between the first and second antennas;
compare the distance to a distance threshold;
determine an angle between the first and second antennas;
compare the angle to an angle threshold; and
select a number of expected ablation zones to overlay on the displayed image based on the operating parameters and the comparisons.

2. The system of claim 1, wherein the controller is further operable to overlay, on the displayed image, one expected ablation zone based on:
the distance being at or less than the distance threshold; and
the angle being at or less than the angle threshold.

3. The system of claim 2, wherein the controller is further operable to overlay, on the displayed image, the one expected ablation zone over the images of the first and second antennas.

4. The system of claim 1, wherein the controller is further operable to overlay, on the displayed image, two expected ablation zones based on:
the distance being at or greater than the distance threshold; or
the angle being at or greater than the angle threshold; or
a combination thereof.

5. The system of claim 4, wherein the controller is further operable to overlay, on the displayed image:
a first of the two expected ablation zones over the image of the first antenna; and
a second of the two expected ablation zones over the image of the second antenna.

6. The system of claim 1, wherein the controller is further operable to:
compare a type of the first ablation probe to a type of the second ablation probe; and
select the number of expected ablation zones to overlay on the displayed image based on the comparison of the types.

7. The system of claim 6, wherein the controller is further operable to overlay, on the displayed image, one expected ablation zone based on:
the distance being at or less than the distance threshold;
the angle being at or less than the angle threshold; and
the type of the first ablation probe being the same as the type of the second ablation probe.

8. The system of claim 6, wherein the controller is further operable to overlay, on the displayed image, two expected ablation zones based on:
the distance being at or greater than the distance threshold;
the angle being at or greater than the angle threshold; or
the type of the first ablation probe being different than the type of the second ablation probe; or
combinations thereof.

9. The system of claim 1, wherein the distance threshold is a maximum distance threshold, and the controller is further operable to:
compare the distance to a minimum distance threshold less than the first distance threshold; and
provide an alert based on the distance being less than the minimum distance threshold.

10. The system of claim 1, wherein the operating parameters comprise an activation time and a power level of the first and second ablation probes.

11. A system, comprising:
a first ablation probe including a first antenna;
a second ablation probe including a second antenna; and
a controller in operable communication with a display and operable to:
receive operating parameters associated with the first and second ablation probes;
display, on the display, images of the first and second antennas in a patient, thereby resulting in a displayed image;
compare a distance between the first and second antennas to a distance threshold;
compare an angle between the first and second antennas to an angle threshold; and
determine a number of predicted ablation zones to overlay on the displayed image based on the operating parameters and the comparisons.

12. The system of claim 11, wherein the controller is further operable to overlay, on the displayed image, one expected ablation zone based on:
the distance being at or less than the distance threshold; and
the angle being at or less than the angle threshold.

13. The system of claim 11, wherein the controller is further operable to overlay, on the displayed image, two expected ablation zones based on:
the distance being at or greater than the distance threshold; or
the angle being at or greater than the angle threshold; or
a combination thereof.

14. The system of claim 11, wherein the controller is further operable to:
compare a type of the first ablation probe to a type of the second ablation probe; and
determine the number of predicted ablation zones to overlay on the displayed image further based on the comparison of the types.

15. The system of claim 14, wherein the controller is further operable to overlay, on the displayed image, one expected ablation zone based on:
the distance being at or less than the distance threshold;
the angle being at or less than the angle threshold; and
the type of the first ablation probe being the same as the type of the second ablation probe.

16. The system of claim 14, wherein the controller is further operable to overlay, on the displayed image, two expected ablation zones based on:
the distance being at or greater than the distance threshold;
the angle being at or greater than the angle threshold; or
the type of the first ablation probe being different than the type of the second ablation probe; or
combinations thereof.

17. The system of claim 11, wherein the operating parameters comprise an activation time and a power level of the first and second ablation probes.

18. A non-transitory computer readable medium storing instructions that, when executed by a processor, cause the processor to:
receive operating parameters associated with first and second ablation probes and second ablation probes;
display, on the display, an image of the first and second antennas in a patient;
determine a distance between the first and second antennas;
compare the distance to a distance threshold;
determine an angle between the first and second antennas;
compare the angle to an angle threshold; and
select a number of expected ablation zones to overlay on the displayed image based on the operating parameters and the comparisons.

19. The non-transitory computer readable medium of claim 18, further storing instructions that, when executed by the processor, cause the processor to overlay, on the displayed image, one expected ablation zone based on:
the distance being at or less than the distance threshold; and
the angle being at or less than the angle threshold.

20. The non-transitory computer readable medium of claim 18, further storing instructions that, when executed by the processor, cause the processor to overlay, on the displayed image, two expected ablation zones based on:
the distance being at or greater than the distance threshold; or
the angle being at or greater than the angle threshold; or
a combination thereof.
